(19) United States
(12) Patent Application Publication
(10) Pub. No.: US 2022/0056399 A1
(43) Pub. Date: Feb. 24, 2022

(54) ISOLATE OF CHAETOMIUM GLOBOSUM AND ITS APPLICATION IN WHEAT GROWTH PROMOTION AND YIELD INCREASE

(71) Applicant: Insitute of Plant Protection, Henan Academy of Agricultural Sciences, Zhenzhou City (CN)

(72) Inventors: Yuli SONG, Zhengzhou City (CN); Fei XU, Zhengzhou City (CN); Junmei WANG, Zhengzhou City (CN); Zixing HAN, Zhengzhou City (CN); Yahong LI, Zhengzhou City (CN); Chaohong FENG, Zhengzhou City (CN)

(21) Appl. No.: 17/368,435

(22) Filed: Jul. 6, 2021

(30) Foreign Application Priority Data
Aug. 19, 2020 (CN) 202010838255.0

(51) Int. Cl. C12N 1/14; A01N 63/30

(52) U.S. Cl. CPC C12N 1/145; A01N 63/30

(57) ABSTRACT

This invention provides an isolate of Chaetomium globosum, which is numbered as 12XP1-2-3, preserved by China General Microbiological Culture Collection Center with CGMCC No. 17183 on Jan. 23, 2019. This invention also provides an application of Chaetomium globosum in wheat growth promotion and yield increase. The results of field trials showed that by coating wheat seeds with Chaetomium globosum 12XP1-2-3, the chlorophyll content of leaves can be increased by 1.9%-14.7% at booting stage, and the wheat yield can be increased by 2.3%-10.9%.

### TECHNICAL FIELD

This invention relates to an isolate of Chaetomium globosum and its application in wheat growth promotion and yield increase.

### BACKGROUND

In terms of agricultural production, there is a high demand on wheat yield. And it is a hot direction of research on promoting wheat growth and increasing yield by seed dressing with microbial agents. Chaetomium sp. is widely distributed in soil and plants. This kind of microorganism can colonize wheat roots and promote the nutrient absorption of plants. At present, there is no report on the application of isolated Chaetomium globosum in wheat growth promotion and yield increase.

### SUMMARY

This invention provides an isolate of Chaetomium globosum 12XP1-2-3, which can be used to promote wheat growth and increase yield. The results of field trails show that by coating wheat seeds with Chaetomium globosum 12XP1-2-3, the chlorophyll content of leaves can be increased at booting stage, and the yield of wheat can also be increased.

An isolate of Chaetomium globosum provided by this invention, which is numbered as 12XP1-2-3, preserved by China General Microbiological Culture Collection Center with CGMCC No. 17183 on Jan. 23, 2019.

The above-mentioned Chaetomium globosum can be used to promote wheat growth and increase yield.

This invention also provides a biocontrol agent containing the Chaetomium globosum, which is prepared by the following steps:

Chaetomium globosum as described in claim 1 is inoculated on Potato Sucrose Agar medium and cultured at 25° C. for 15 days. The spore suspension is obtained by surface washing and coating with a glass spreading rod by adding sterile water, and then filtrated with four layers of sterile gauze. The concentration of spore suspension is 1-2×10⁷ ascospores per milliliter, which is the first-degree species containing Chaetomium globosum;

The spore suspension is inoculated on sterilized wheat grains and cultured for 15 days until grains are covered with hyphae and spores. Then the second-degree species containing Chaetomium globosum is obtained after rinsing and isolating.

The biocontrol agent is then obtained by dispersing the second-degree species containing Chaetomium globosum into sodium carboxymethyl cellulose solution.

Preferably, the mass fraction of the sodium carboxymethyl cellulose solution is 4%; and the volume ratio of the spore suspension to the sodium carboxymethyl cellulose solution is 3:1.

Preferably, the wheat grains are soaked in water for 12 h, sterilized for 1 h, placed for 1-2 days, and sterilized again for 30 min.

The biocontrol agent can be used to promote wheat growth and increase yield, and the wheat seeds are coated with 5.0-5.3×10⁴ ascospores per seed.

Instructions on Biological Preservation Biological material: Chaetomium globosum 12XP1-2-3; taxonomic name: Chaetomium globosum; preserved on Jan. 23, 2019 by China General Microbiological Culture Collection Center, which is located at Institute of Microbiology, Chinese Academy of Sciences, No. 3, Yard 1, Beichen West Road, Chaoyang District, Beijing, and its preservation number is CGMCC No. 17183.

Compared with the prior art, the beneficial effects of this invention are as follows:

For the Chaetomium globosum 12XP1-2-3 provided by this invention, the results of field trials show that by coating wheat seeds with Chaetomium globosum 12XP1-2-3, the chlorophyll content of leaves can be increased at booting stage, and the yield of wheat can also be increased.

### BRIEF DESCRIPTION OF THE DRAWINGS

FIGS. 1a and b show the growth effect of wheat at jointing stage;

FIGS. 2a and b show the growth effect of wheat at grain filling stage; Where, a is the wheat not coated with Chaetomium globosum 12XP1-2-3, b is the wheat coated with Chaetomium globosum 12XP1-2-3.

### DETAILED DESCRIPTION OF THE PREFERRED EMBODIMENTS

This invention is further described in detail below in combination with specific embodiments for understanding, rather than limiting, the scope of protection of this invention.

### Embodiment 1

Isolation of Chaetomium globosum 12XP1-2-3

At the filling stage of wheat, healthy plants were collected from the wheat field in Xiping County of Henan Province and put into paper bags. The samples were stored in a refrigerator at 4° C. Wheat roots were rinsed, cut into 2-3 cm sections and sterilized with 75% ethanol for 10-30 s and 1% NaClO for 1.5 min. Root sections were rinsed with sterile water for 3 times, dried with sterile filter papers and cut into 0.5 cm-long sections. Then they were placed on potato dextrose agar medium (PDA, with raw materials of: peeled potato 300 g, glucose 20 g, agar 20 g and distilled water 1,000 ml). After 5-7 days culture at 20° C., marginal hypha was picked up with sterile forceps, placed on a new PDA plate and cultured for 5-7 days under the same condition as above. Then mecelium with uniform colonies was picked up, cultured on PDA slant medium and stored at 4° C.

### Embodiment 2

DNA Extraction of Chaetomium Globosum 12XP1-2-3

Chaetomium globosum 12XP1-2-3 was cultured on PDA for 5-7 days at 20° C. and then five hyphae blocks from the edge were picked up and placed evenly on the PDA plate covered with sterilized cellophane. 3-5 days later, hyphae was scraped with a sterilized shovel, freezed with liquid nitrogen, and stored in a refrigerator at −20° C. For DNA extraction, 20-25 mg hypha was put into a 1.5 mL EP pipe which was added with a little liquid nitrogen and ground into flour with a pre-cooled iron nail. After being added with 500 μl Extraction Buffer (50 mM Tris-Cl (pH 8.0), 150 mM NaCl, 100 mM EDTA (pH 8.0)), the mixture was oscillated and suspended with a vortex mixer, then added with 25 μl preheated sodium dodecyl sulfonate (SDS), blended reversedly and kept in a water bath at 37° C. for 1-3 h. Afterwards, the pipe was added with 75 μl NaCl (5 M) and blended reversedly, followed by being added with 65 μl CTAB/NaCl (10% CTAB, 0.7 M NaCl) solution and kept at 65° C. for 30 min. A mixture of Tris-phenol, chloroform and isoamylol (25:24:1) with equal volume of 700 μl was added to the pipe, prior to blending and centrifugation at 1000 rpm and 4° C. for 10 min. The supernatant (550 μl) was transferred to another EP pipe, precipitated by adding pre-cooled isopropanol (330 μl) and kept at −20° C. for 10 min, and then centrifuged at 10000 rpm and 4° C. for 10 min. The supernatant was abandoned. The precipitate was washed twice by adding 70% ethanol, dried in a workbench for 5-10 min, and dissolved in sterile ddH₂O for storage at −20° C.

### Embodiment 3

Molecular Identification of Chaetomium Globosum 12XP1-2-3

In this experiment, primers ITS1 (5′-TCC GTA GGT GAA CCT GCG G-3′) and ITS4 (5′-TCC TCC GCT TAT TGA TAT GC-3′) were used to amplify DNA fragments containing ITS1, 5.8S rDNA and ITS2 of the tested strain. The reaction system is determined with reference to Daval et al. (2010). PCR reaction program: 3 min at 95° C.; 45 s at 95° C., 30 s at 50° C., 1 min at 72° C. for 35 cycles; and 10 min at 72° C. The PCR products were sequenced and analyzed by BLAST sequence alignment in NCBI. The sequence alignment results showed that the biocontrol fungi 12XP1-2-3 was Chaetomium globosum.

TABLE 1
PCR Reaction System

| Composition | Concentration | Dosage (μl) |
|---|---|---|
| 10× PCR buffer solution | 10 mM | 5.0 |
| dNTP mixture | 10 mM | 1.0 |
| TaqDNA polymerase | 5 u | 0.5 |
| Primer 1 | 20 μM | 1.0 |
| Primer 2 | 20 μM | 1.0 |
| DNA template | 10-50 ng/μl | 1.0 |
| Double-distilled water | Pure water | 40.5 |
| Total volume (μl) | | 50.0 |

### Embodiment 4

Effects of Seed Coating with Chaetomium Globosum 12XP1-2-3 on Chlorophyll Content in Wheat Leaves at Booting Stage

A SPAD502 chlorophyll meter was used to measure the content of chlorophyll in flag leaf and top second leaf of 10 seedlings at each of the 3 points selected from the seed coating group and blank control group (that is, the wheat seeds in these areas were not coated with 12XP1-2-3 biocontrol agent).

Growth rate of chlorophyll=(content of chlorophyll in flag leaf or top second leaf from the seed coating group by Chaetomium globosum 12XP1-2-3−content of chlorophyll in flag leaf or top second leaf from the blank control group)/content of chlorophyll in flag leaf or top second leaf from the blank control group.

The results are shown in Table 2.

TABLE 2
Effects of Chaetomium Globosum 12XP1-2-3 on
Chlorophyll Content of Wheat Leaves at Booting Stage

| | Chlorophyll content | | Chlorophyll content | |
|---|---|---|---|---|
| | of ″AK 58″ | | of ″AK 58″ | |
| | at booting stage in | | at booting stage in | |
| | Wenxian County from | | Kaifeng from 2018 to | |
| | 2018 to 2019 (SPAD) | | 2019 (SPAD) | |
| | | Top Second | | Top Second |
| Description | Flag Leaf | Leaf | Flag Leaf | Leaf |
| Blank control | 43.2 | 56.2 | 40.7 | 52.6 |
| group | | | | |
| 12XP-2-3 | 51.5 | 56.2 | 46.7 | 56.2 |
| Growth rate of | 1.9 | 0 | 14.7 | 6.8 |
| chlorophyll (%) | | | | |

It can be seen from Table 2 that the content of chlorophyll in flag leaf of wheat treated with seed coating of Chaetomium globosum 12XP1-2-3 increased by 1.9%-14.7% compared with the blank control group, and the content of chlorophyll in top second leaf increased by 0-6.8%.

### Embodiment 5

Effect of Chaetomium Globosum 12XP1-2-3 on Wheat Yield Increase in Field

1. Seed Coating with Chaetomium globosum

Chaetomium globosum 12XP1-2-3 was activated on fresh PDA, and then propagated on 100 PDA culture dishes with 9 cm in diameter. 15 days later, perithecium was coated and grinded with a glass spreading rod by adding sterile water, and the ascospore suspension was kept at about 100 ml with the concentration of 1.0-2.0×10⁷ spores/ml, which is the first-degree species containing Chaetomium globosum. Then 20 ml of spore suspension was inoculated into a sterilized stainless steel tray which is 60 cm long, 40 cm wide and 7 cm high and filled with sterilized wheat grains with the thickness of 2 cm. Wheat grains were soaked in water for 12 h, sterilized for 1 h, placed for 1-2 days, and sterilized again for 30 min. 15 days later grains were covered with hyphae and spores, After rinsing with water, the second-degree species containing Chaetomium globosum was obtained (If the concentration is too low, centrifuge it at 3,000 rpm for 5 min, then remove water and resuspend it). Then 75 ml of second-degree species was mixed with 25 ml of 4% carboxymethyl cellulose salt solution to blend into 100 ml fungi solution (1.0×10⁷ spores/m1), which was then added to 5 kg wheat seeds and mixed evenly. By washing the seeds and spread plate, the number of spores on the surface of coated seeds with Chaetomium globosum 12XP1-2-3 was determined as 5.0-5.3×10⁴ ascospores per seed.

2. Data Investigation and Recording

Field sampling: determination of wheat yield at maturity stage. Yield increase rate=(yield from seed coating with Chaetomium globosum 12XP1-2-3−yield from blank control group)/yield from blank control group. The results are shown in Table 3.

TABLE 3
Effect of Seed Coating on Wheat Yield Increase

| | Yield/Mu (kg) | | | |
|---|---|---|---|---|
| | | ″AK 58″ in | ″BN 207″ in | |
| | ″BN 207″ in | Wen County | Wen County | ″BN 207″ in |
| | Kaifeng in | in | in | Anyang in |
| Description | 2017-2018 | 2018-2019 | 2019-2020 | 2019-2020 |
| Blank | 290.2 | 559.8 | 700.5 | 449.0 |
| control | | | | |
| group | | | | |
| 12XP1-2-3 | 297.0 | 621.0 | 762.0 | 424.0 |
| Yield | 2.3 | 10.9 | 8.8 | 5.9 |
| increase rate | | | | |
| (%) | | | | |

It can be seen from Table 3 that the wheat yield from seed coating with Chaetomium globosum 12XP1-2-3 increased by 2.3%-10.9% compared with the blank control group.

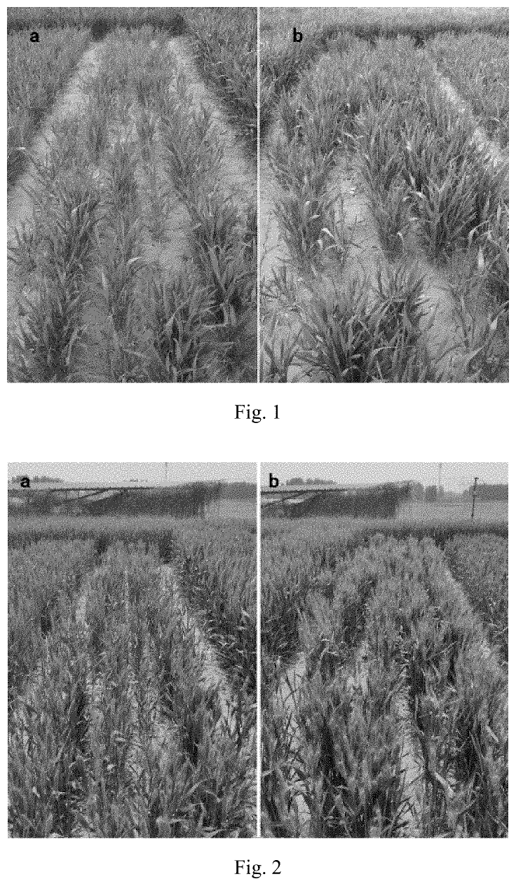

As shown in FIG. 1 and FIG. 2, FIG. 1 showed the growth effect of wheat at jointing stage, and FIG. 2 showed the growth effect of wheat at grain filling stage; wherein, “a” represented the wheat not coated with Chaetomium globosum 12XP1-2-3, and “b” represented the wheat coated with Chaetomium globosum 12XP1-2-3. It can be seen from FIGS. 1-2 that the wheat grows better after being coated with Chaetomium globosum 12XP1-2-3 provided by this invention.

It should be noted that the steps and methods adopted in the claims of the present invention are the same as those in the above-mentioned embodiments. In order to prevent repetition, the preferred embodiments are described in the present invention. However, once those skilled in the art get to know the basic inventive concept, they may make additional changes and modifications to these embodiments. Therefore, these appended claims are intended to be interpreted to include preferred embodiments and all changes and modifications falling within the scope of the present invention.

Obviously, those skilled in the art can make various changes and variations to this invention without deviating from its spirit and scope. Thus, if these modifications and variations of the invention fall within the scope of the claims of the invention and their equivalents, the invention is also intended to include these modifications and variations.

1. An isolate of Chaetomium globosum, which is numbered as 12XP1-2-3, and preserved by China General Microbiological Culture Collection Center with CGMCC No. 17183 on Jan. 23, 2019.

2. An application of Chaetomium globosum in wheat growth promotion and yield increase according to claim 1.

3. A biocontrol agent, containing the Chaetomium globosum of claim 1.

4. The biocontrol agent according to claim 3, prepared in the following manner:
the isolate of Chaetomium globosum is inoculated on Potato Sucrose Agar medium and cultured at 25-° C. for 15 days; a spore suspension is obtained by surface washing and coating with a glass spreading rod by adding sterile water, and then filtrated with four layers of sterile gauze; wherein the concentration of spore suspension is 1-2×10⁷ ascospores per milliliter, which is a first-degree species containing Chaetomium globosum;
the spore suspension is inoculated on sterilized wheat grains and cultured for 15 days until grains are covered with hyphae and spores, then a second-degree species containing Chaetomium globosum is obtained after rinsing and isolating; and
the biocontrol agent is then obtained by dispersing the second-degree species containing Chaetomium globosum into sodium carboxymethyl cellulose solution.

5. The biocontrol agent according to claim 4, wherein the mass fraction of the sodium carboxymethyl cellulose solution is 4%; and the volume ratio of the spore suspension to the sodium carboxymethyl cellulose solution is 3:1.

6. The biocontrol agent according to claim 4, wherein the sterilized wheat grains are soaked in water for 12 h, sterilized for 1 h, placed for 1-2 days, and sterilized again for 30 minutes.

7. An application of the biocontrol agent according to claim 3, in wheat growth promotion and yield increase.

8. The application according to claim 7, wherein the wheat seeds are coated with 5.0-5.3×10⁴ ascospores per seed.
